(19) United States
(12) Patent Application Publication
(10) Pub. No.: US 2008/0039676 A1
(43) Pub. Date: Feb. 14, 2008

(54) Means and method for marking human tissue that may be malignant

(76) Inventors: Robert E. Fischell, Dayton, MD (US); David J. Mullen, Old Greenwich, CT (US)

Correspondence Address: Dr. Robert E. Fischell, 14600 Viburnum Drive, Dayton, MD, 21036, US

(21) Appl. No.: 11/805,399

(22) Filed: May 24, 2007

Related U.S. Application Data
(63) Continuation-in-part of application No. 11/502,906, filed on Aug. 11, 2006.

(51) Int. Cl. A61M 37/00

(52) U.S. Cl. 600/12

(57) ABSTRACT

Disclosed herein is a system for placing an elongated permanent magnet into a volume of tissue within a human body, including placement following a large core needle tissue biopsy procedure, where the tissue is suspected of being cancerous. The magnet's position is then detected by a magnet locating system that is used by a surgeon to find and remove the suspected tissue volume including a margin of tissue to assure that any and all cancer cells have been excised.

### REFERENCE TO A PRIOR PATENT APPLICATION

This is a continuation-in-part application of the U.S. patent application Ser. No. 11/502,906 filed on Aug. 14, 2006.

### FIELD OF USE

This invention is in the field of methods and devices for marking human tissue that may be cancerous so that tissue can be excised by a surgeon.

### BACKGROUND OF THE INVENTION

The need for accurate preoperative image guided localization of nonpalpable breast lesions has been well described, and the frequency of use for this technique is increasing. Not only mammographically detected lesions require localization, but also lesions that may be found by any other imaging technique such as ultrasound, MRI, nuclear medicine or other technologies not yet described. Such localizations generally require the positioning of a temporary marker, most frequently constructed of a metal anchor on the end of a wire inserted through a needle that has been accurately positioned by image guidance prior to the release of the marker. See, Frank H. A., Hall F. M., Steer M. L., Preoperative Localization of Nonpalpable Breast Lesions Demonstrated by Mammography; New England Journal of Medicine, 1976; 296:259-260. In addition, the implantation of a small metal “clip” marker following large core biopsy under image guidance may be used when the visualized target has been substantially removed during the diagnostic procedure (thus compromising future successful localization). See, Burbank F. et al., “Tissue Marking Clip for Stereotactic Breast Biopsy: Initial Placement Accuracy, Long-term Stability, and Usefulness as a Guide for Wire Localization”; Radiology, 1997; 205:407-415.

The need for such localization is best understood in the breast but will be of growing importance in other organ systems particularly for marking suspected tissue volume in the lungs. The explosive growth of diagnostic imaging has increased the frequency of detection of small lesions throughout the body that cannot be seen or felt by the surgeon who is charged with the task of removing the suspected tissue volume. Guidance for radiation therapy or other emerging ablation techniques using thermal, laser, radiofrequency or other methods of local energy or drug deposition to kill cells is also needed.

Many metal devices to accomplish breast marking or localization have been devised (e.g., U.S. Pat. Nos. 4,799,495; 5,011,473; 5,057,085; 5,083,570; 5,127,916; 5,158,084; 5,221,269; 5,234,426; 5,409,004; 5,556,410; 6,053,925; and 6,544,269). These devices all have the significant limitation of requiring the image guided localization procedure immediately before the surgery. Because the anchoring device is connected to a wire that protrudes through the skin, it must be promptly removed. Even those devices now approved for implantation into the breast (following the special case of a small lesion which has been substantially removed during image guided large core needle biopsy) must be re-localized with a second temporary device on the day of definitive surgery. The need for immediate preoperative localization frequently creates logistical problems for radiology departments and operating room personnel. Any system that could eliminate the need for prompt surgery after localization of the suspected tissue volume would be an advance in this field.

Described herein are alternative embodiments of the permanent magnet marker design and also a means and method to address the limitation in those devices now approved for implantation into the breast following the special case of a small lesion which has been substantially removed during image guided large core needle biopsy. Prior art devices to mark the general area of the breast from which a large core needle biopsy tissue sample has been obtained require re-localization with a second temporary device on the day of definitive surgery. Any system that could eliminate the need for a second localization procedure of the already marked suspected tissue volume would be an advance in this field. The need for immediate preoperative localization not only creates logistical problems for radiology departments and operating room personnel, but also subjects patients to addition physical discomfort and psychological stress.

Although some have attempted solutions to these problems, a simple and cost effective approach has not yet been found. Thus, a device that could be implanted by a radiologist at one time after the completion of a large core needle biopsy and then independently removed by a surgeon at another time with no further patient preparation if a subsequent surgical procedure is needed would represent an advancement in the field and prove to be a useful adjunct to the method described in U.S. patent application Ser. No. 11/502,906 “Means and Method for Marking Tissue in a Human Subject” by R. E. Fischell and D. J. Mullen.

Before the era of diagnostic breast imaging, only palpable lesions were detectable. Palpable lesions can be biopsied by a surgeon without any form of marking or guidance other than physical examination of the breast before and during the surgical procedure. Lesions that are detectable only by imaging, however, are best biopsied after marking. Although this is now done following wire localization, some have suggested the use of markers that may render a previously nonpalpable lesion palpable, thus providing the surgeon with a familiar method of tactile guidance. See, Debbas, Apparatus for Locating a Breast Mass, U.S. Pat. No. 5,662,674; Fulton et al. Biopsy Localization Method and Device, U.S. Pat. No. 6,730,042; and Fulton et al. Target Tissue Localization Device and Method, U.S. Pat. No. 6,409,742. These proposed devices all have one significant drawback in common: they are large and may be expected to be uncomfortable for patients. This problem may be further compounded when these devices must remain in position for some length of time.

In U.S. patent application Ser. No. 11/660,205, D. J. Mullen describes a means for the surgeon to find a suspected tissue volume using a metal detector that detects wires that have been implanted within the suspected tissue volume. Although this system is workable, the metal detector for such small wires can only detect them within a comparatively close range of the suspected tissue volume.

In U.S. Pat. No. 6,698,433, D. N. Krag describes several means to localize a suspected tissue volume. These methods include the placement of a multiplicity of tiny magnets around the suspected tissue volume. Specifically, in FIG. 1 of the Krag patent, six small objects that could be permanent magnets are located around the suspected tissue volume. In FIG. 1A, ten small objects that could be permanent magnets are placed around a suspected tissue volume. In FIG. 2b of the Krag patent he shows a tiny magnet which has a diameter that is actually greater in its transverse dimension as compared to the length of the magnet between the magnetic poles. That is, the ratio of the distance between the poles compared to the magnet diameter is less than 1.0. Such tiny magnets have an extremely small field at any reasonable distance outside of a human breast because the close proximity of the north and south poles result in cancellation of the magnet's external field. A collection of six to ten such magnets randomly placed around a suspected tissue volume would not be at all detectable using a magnetometer probe outside of the breast. This is in contradistinction to a single (or even two or three) elongated magnets that have a magnetic field that would be readily detectable outside of the breast. What is required but not at all anticipated by the teachings of the Krag patent is an elongated magnet that has a distance between the poles that is at least 1.5 times the magnet's diameter and preferably at least 3 to 10 times the magnet's diameter.

### SUMMARY OF THE INVENTION

Disclosed herein is a system for placing an elongated permanent magnet into a volume of tissue within a human body, including placement following a large core needle tissue biopsy procedure, where the tissue is suspected of being cancerous. The magnet's position is then detected by a magnet locating system that is used by a surgeon to find and remove the suspected tissue volume including a margin of tissue to assure that any and all cancer cells have been excised.

A first method to accomplish the placement of the magnet is to use a magnet injector that has a sharpened cannula with the magnet placed at the cannula's distal end. One of several different methods can be used by a radiologist to determine the location of the suspected tissue volume. These methods include sonography, fluoroscopy, MRI and mammography. After the injector places the magnet within the suspected tissue volume, a spring release mechanism is pulled out of the handle of the injector and a spring within the handle quickly removes the cannula from the breast. This leaves the magnet in place for detection by a magnetometer probe that is operated by a surgeon. A preferred embodiment of the system is that the magnet extends beyond the extremities of the suspected tissue volume by a length that is approximately equal to the tissue margin that the surgeon hopes to attain. Thus when the magnet is removed, the suspected tissue volume plus the additional margin tissue are also removed.

Locating the magnet within the breast is accomplished by means of a magnetometer probe that can take several different forms. One form is a total field magnetometer located near the distal end of the probe. Another embodiment of the magnet locating system is to use a vector magnetometer or a vector magnetic gradient detector. Such a gradient detector has the advantage of being able to automatically cancel out the earth's magnetic field and any local field whose gradient is (as would be expected) much smaller than the gradient of the magnetic field from the implanted magnet.

To assist the surgeon in detecting the location of the magnet, it is desirable to place the magnet so as to be parallel to the patient's chest wall (i.e., horizontal) with the patient lying down and with the orientation of the long axis of the magnet being known to the surgeon, for example with the long axis of the magnet being perpendicular to the body's long axis. This is particularly valuable when a vector magnetic gradient magnetometer is used to detect the position of the magnet within the breast.

A second method for placing the magnet within the suspected tissue volume is to first use a sharp stylet within a cannula needle to penetrate through the suspected tissue volume. The stylet is then removed from the cannula needle that is placed through the suspected tissue volume. A magnet injector with a squared off distal end of its cannula and having the magnet at the injector cannula's distal end is then placed within the cannula needle that is already placed through the suspected tissue volume. The cannula needle is then pulled out of the breast. The elongated magnet within the cannula of the injector is then centered within the suspected tissue volume. The spring release handle is then pulled out of the handle of the magnet injector and a spring within the handle of the magnet injector pulls the cannula out of the breast leaving the magnet in place through the suspected tissue volume.

It is also envisioned to first place the tip of the insertion needle (which is also the distal tip of the magnet) at the center of the suspected tissue volume. The next step would use a spring release to quickly advance the tip of the needle by half the length of the magnet and then use a second spring release to pull back the needle so that the magnet would lie centered in the suspected tissue. It is believed that this technique could more accurately place the permanent magnet at the center of the suspected tissue volume.

Described herein are a means and method for placing a permanent magnet within the suspected tissue volume by placing it through the inner lumen of a large core needle biopsy device, which has been first used to take a sample of breast tissue. A flexible, magnet insertion cannula containing an elongated magnet marker with an anchoring mechanism compressed inside of the cannula is positioned through the inner lumen of the biopsy device which has just been used to take a sample of a suspected tissue volume. The magnet marker is positioned by using a plunger to push the magnet out of the magnet insertion cannula through an opening at the end of the magnet insertion cannula. Upon exiting the magnet insertion cannula, the compressed anchoring mechanism of the magnet will deploy, thereby permanently fixing the magnet in place within the center of the biopsy cavity. By using the inner lumen of a needle biopsy device, which has just been used to take a sample of a suspected tissue volume, no further instrumentation is needed and no additional trauma to the breast takes place. Further, the use of the already positioned biopsy device will insure that the position of the elongated magnet marker will correspond precisely to the location of the tissue just removed.

Although one use of the means and method described herein is for marking tissue following large core breast biopsy, this means and method will serve equally well following the use of any hollow instrument that is used to obtain a tissue sample from anywhere within the human body, for example following thoracoscopic, laparoscpic or endoscopic biopsies or surgeries. Further, a magnet locating system may be easily placed on or through a thoracascope, laparascope or endoscope to aid a physician in the detection of a previously marked suspected tissue volume. Thus the concepts described herein for marking and detecting a suspected tissue volume in a breast can also be applied to other regions of the body such as the lungs or other human tissue that may be cancerous.

Thus one object of the present invention is to use a single, elongated permanent magnet to mark the location of a suspected tissue volume that may be cancerous.

Another object of this invention is to use a magnetometer probe to detect the position of the magnet within the suspected tissue volume.

Still another object of this invention is to use a vector magnetic gradient magnetometer to detect the position of the magnet so as to negate the effect of the earth's magnetic field and any local magnetic field.

Still another object of this invention is to use an elongated magnet that extends beyond the boundaries of the suspected tissue volume by a length approximately equal to the margin of normal tissue that should be removed around the suspected tissue volume to assure that no malignant cells remain in the body.

Still another object of this invention is to use a wireless probe system so that the probe can be operated by the surgeon without the encumbrance of a wire that connects the probe to the visual or audio means that indicates to the surgeon that he/she is approaching the implanted magnet.

Still another object of this invention is to have the entire magnetic field detection system be contained within a hand held probe that is used by the surgeon to locate the implanted magnet thus obviating the need for either wired or wireless connections for the magnet probe system.

Still another object of this invention is to place a magnet marker through a biopsy device after the tissue biopsy is performed and before the biopsy needle is removed from the breast to mark the location of the suspected tissue volume.

Still another object of this invention is to use the systems and methods described herein to place a magnet into any suspected tissue volume within a human body that may need to be excised by a surgeon.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a longitudinal cross section of a magnet injector that is used to place an elongated magnet within human tissue that may be cancerous.

FIG. 2 is a plan view of an anchor that is attached to the end of the magnet.

FIG. 3 is a transverse cross section of the magnetic injector handle at section 3-3 of FIG. 1 showing the means to release the spring that ejects the cannula from the breast.

FIG. 4 illustrates the magnetic field of a magnet placed into a female breast at the site of a suspected tissue volume. FIG. 4 also shows a magnet locating system for detecting the position of the magnet within the breast.

FIG. 5 shows a stylet and cannula needle system that is placed through a suspected tissue volume.

FIG. 6 shows a magnet injector placed within the cannula that was placed through the breast after the stylet was removed.

FIG. 7 shows the cannula of the insertion needle having been removed from the breast and slid along the cannula of the magnet injector.

FIG. 8 shows the magnet centered around the suspected tissue volume within the breast with the magnet anchors deployed.

FIG. 9 illustrates a permanent magnet located near the center of the type of wire that is currently used to mark a suspected tissue volume within the breast.

FIG. 10A is a longitudinal cross section of an alternative embodiment of a magnet marker showing the pre-deployed configuration of the magnet marker that has a metal center anchor.

FIG. 10B is a longitudinal cross section of the magnet marker of FIG. 10A in its deployed state.

FIG. 11A is a longitudinal cross section of another alternative embodiment of the magnet marker that uses an expandable, sponge-like center anchor shown in its pre-deployed state within a cannula.

FIG. 11B is a longitudinal cross section of the magnet marker of FIG. 11A shown with the sponge-like center anchor in its deployed state.

FIG. 12 is a longitudinal cross section of a distal portion of a biopsy needle device that is used for needle biopsy procedure and also showing another embodiment of the magnet marker located within the biopsy needle.

FIG. 13 shows the magnet marker of FIG. 12 in its deployed form within the breast of a human female.

### DETAILED DESCRIPTION OF THE INVENTION

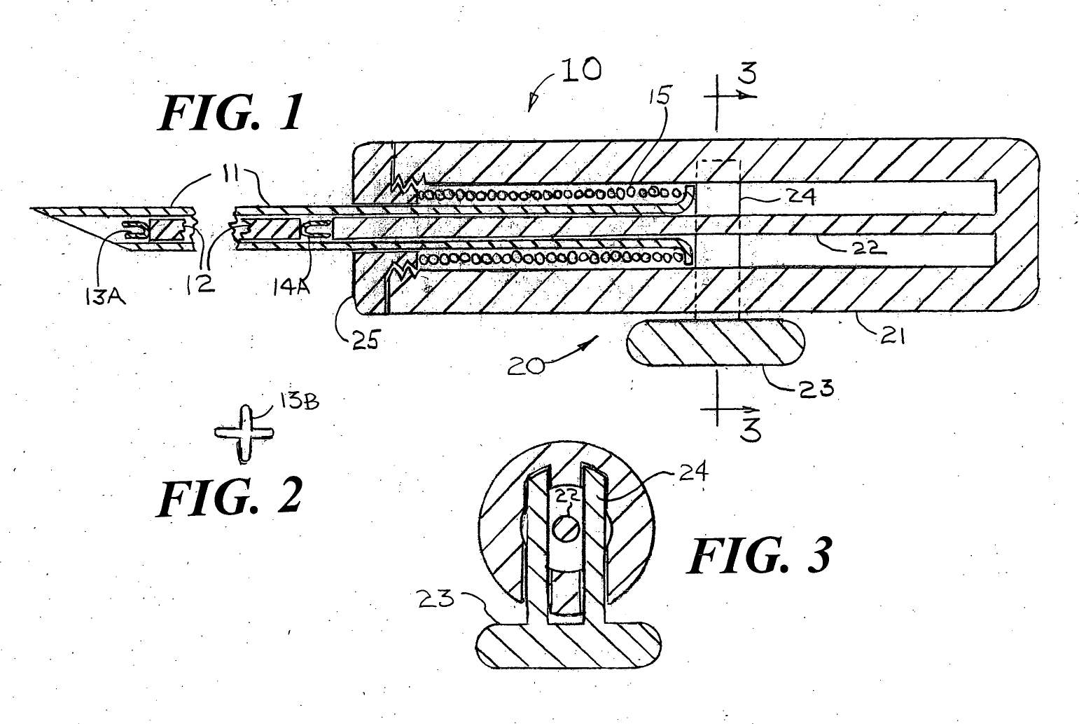

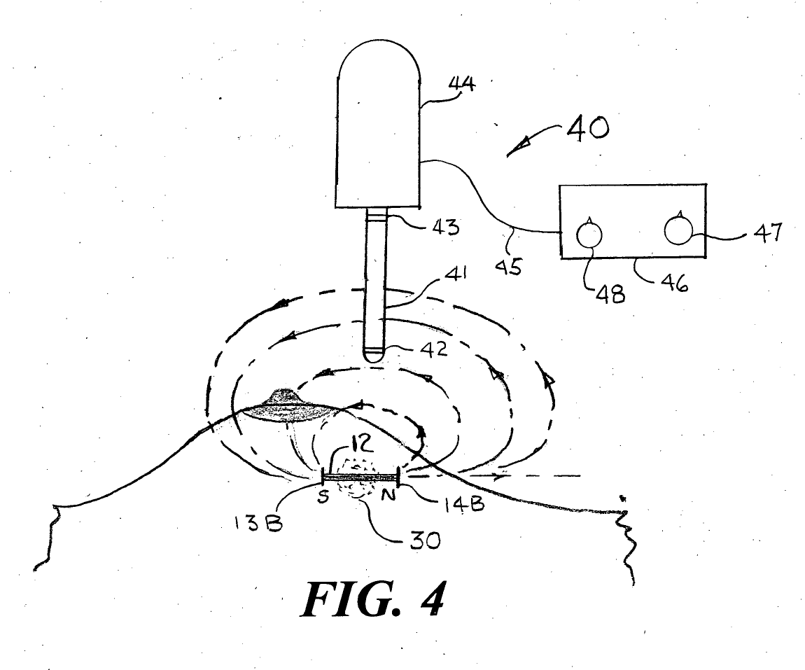

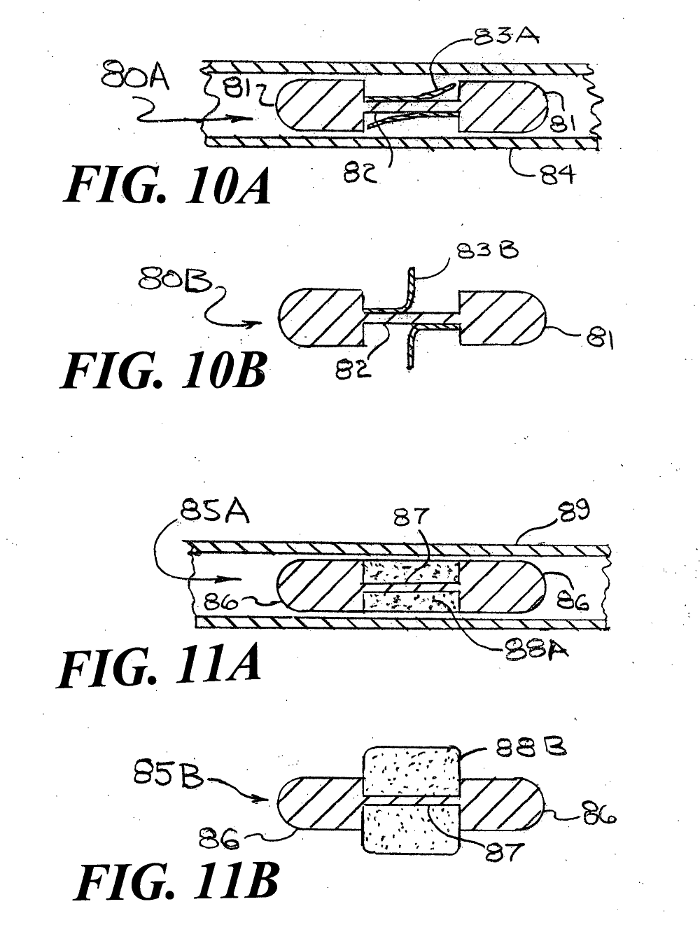

FIG. 1 shows a magnet injector 10 having a cannula 11 into which is placed a permanent magnet 12 having a pre-deployed distal anchor 13A fixedly attached at the magnet's distal end and a pre-deployed proximal anchor 14A fixedly attached at the magnet's proximal end. As shown in FIGS. 10A, 10B, 11A, 11B, 12 and 13, it is also conceived that the magnet could have one or more anchors deployed near the center of the magnet that also would be used to stabilize the position of the magnet within the breast. The shape of the deployed distal anchor 13B is shown in FIG. 2. The cannula 11 is positioned within a handle 20 that has an outer cylinder 21, an inner cylinder 22 and a spring release handle 23 that is attached to a spring holder 24 that keeps the spring 15 from expanding until the magnet 12 is ready for release within the suspected tissue volume. The cross section at 3-3 of FIG. 1 is shown as FIG. 3 that provides additional details of the spring release handle 23, the spring holder 24 and the inner cylinder 22. The distal end of the spring 15 is held by the end cap 25 and the proximal end of the spring is held by an expanded end section of the cannula 11. When the magnet 12 is placed into a suspected tissue volume 30 (see FIG. 4), the spring release handle 23 is pulled out of the outer cylinder 21 which removes the spring holder 24 which causes the cannula 11 to be pulled into the handle 20 while the inner cylinder 22 causes the magnet 12 to be accurately retained within the suspected tissue volume 30 as shown in FIG. 4.

The cannula 11 would typically be made from thin-walled stainless steel with an inside diameter that typically would be just slightly larger than 1.0 mm. A range of diameters for the cannula 11 from 0.3 to 3.0 mm is certainly possible for this application. The magnet 12 would be made from any permanent magnetic material having a reasonably high-energy product. A preferred magnetic material would be strong and ductile. A preferred permanent magnet material would be an alloy that is comparatively flexible and machinable such as Vicalloy or Arnokrome 3.

FIG. 4 shows a human breast into which the magnet 12 has been placed into the suspected tissue volume 30 by the injector 10 of FIG. 1 or the injector 60 of FIG. 6. FIG. 4 also shows the deployed anchors 13B and 14B. The anchors 13B and 14B are optimally formed from a shape memory alloy such as Nitinol. Optimally, the Nitinol would have a transition temperature of about 90 degrees F. so that it could be welded or otherwise fixedly attached to the ends of the magnet 12 and then bent so as to be easily placed within the cannula 11. After the predeployed anchors 13A and 14A of FIG. 1 are placed into breast tissue, the temperature of the breast will cause the anchors to deploy to the shape that is shown in FIGS. 2 and 4. The function of these anchors is to prevent shifting of the magnet within the breast after it has been accurately placed within the suspected tissue volume 30. In order to get a reasonably high level of magnetic field outside of the breast, it is necessary for the magnet 12 to have a reasonably high energy product and also that the magnet 12 have a reasonably long length for the distance between the magnetic north pole, N and the south magnetic pole, S. This ratio of length between the poles to the diameter of the magnet 12 should be at least 1.5:1 and preferably in the range of at least 5:1. It is ideal for the magnet 12 to protrude beyond the suspected tissue volume 30 by at least 2 mm so that there will be a margin of normal tissue between the perimeter of the suspected tissue volume and the end of the magnet 12. An optimal size for the magnet 12 would have it extend at each of its ends for approximately 5 mm beyond the outer surface of the suspected tissue volume 30. Therefore, the magnet 12 should be at least 10 mm long to provide an optimum margin for the very smallest suspected tissue volume 30. The surgeon who would be removing the magnet 12 and the suspected tissue volume 30 would be guided by the extremities of the magnet 12 for the removal of tissue with an adequate margin. Specifically, the surgeon should remove a volume of tissue that has a spherical diameter that is approximately equal to the distance between the deployed anchors 13B and 14B. In point of fact, since most of the suspected tissue volumes found by mammography are very small, and since removing a spherical tissue volume with a diameter of 20 mm is quite small and not disfiguring, a length of 20 mm for the magnet 12 would be ideal for most applications. If the suspected tissue volume 30 is not at all spherical in its extent, the surgeon could be advised of that shape so that the appropriate tissue excision could be accomplished. It is also conceived that 2 or 3 elongated magnets could be used to define a suspected tissue volume that is not small and is of an odd shape. However, the typical suspected tissue with modern mammography is sufficiently small so that a spherical excision of tissue to the diameter which is essentially the length of the magnet 12 will be generally sufficient to maintain an adequate tissue margin.

The use of the deployed anchors 13B and 14B of FIG. 4 represents a unique aspect of the invention to provide centering of the magnet 12 with respect to the tissue volume 30 under varied deployment conditions. Needle localization is generally accomplished using mammographic guidance. The use of stereotactic needle localization has been described, but carries with it certain significant limitations. These limitations are predominantly due to the fact that, although accurate localization within millimeters of the target is readily accomplished while the breast is compressed within the stereotactic apparatus, upon release of compression significant errors in the depth of marker placement are frequently encountered. This limitation has been understood as the so called “accordion” effect and is due to the re-expansion of the breast after the release of compression and often results in difficulty maintaining precise localization of a marker after the release of compression. In the first embodiment of the present invention this limitation is overcome, since the presence of deployed anchors 13B and 14B (of FIGS. 4 and 8) at the distal as well as the proximal ends of the magnet 12 will prevent both forward and reverse migration of the magnet 12 with respect to the tissue volume 30. The embodiments shown in FIGS. 10A, 10B, 11A, 11B, 12 and 13 show center anchors that can be even more effective in maintaining the accurate positioning of a magnet marker in the breast or other human tissue. By utilizing end or center anchors, accurate localization will be reliably maintained after the breast is released from compression. Thus, an advantage of a device according to the present invention is that it will allow accurate tissue localization to be easily accomplished using stereotactic mammography equipment, significantly increasing the range and number of procedures that can be performed on stereotactic mammography equipment. The stereotactic mammography equipment itself, as well as the rooms in which this equipment is located, represents a significant capital expense for a facility offering such procedures. Allowing localizations to be performed on such equipment enhances the productivity of these rooms. Furthermore, patients will experience significant benefits in the use of this embodiment of the inventive procedure. It is well known that the prone position offered by stereotactic mammography improves patients' tolerance of needle procedures in the breast by decreasing the incidence of vasovagal responses as well as the perception of pain. Procedure time is significantly decreased by the use of digital receptors and precision is improved by the stereotactic technique.

FIG. 4 also shows a magnet locating system 40 that includes a magnetometer probe 41, an end magnetic field detector 42, a reference magnetic field detector 43, and a handle 44 that is connected by a wire 45 to the electronic module and range indicator 46. The end magnetic field detector 42 can be either a total field magnetometer or a vector magnetometer that would measure only one component of the magnetic field from the implanted magnet 12. If the end magnetic field detector 42 and the reference magnetic field detector 43 are each total field magnetometers, then the magnet locating system 40 would have a means to null out the earth's field and any local magnetic field where the patient is situated. If a single axis, vector magnetometer is used, then the reference detector 43 must also be a vector magnetometer that measures the magnetic field intensity in the same direction as the end magnetic field detector 42. The preferred embodiment of the magnet locating system 40 would utilize the detectors 42 and 43 as a vector magnetic gradiometer which measures the difference in the vector magnetic field intensity at the position of the detector 42 compared to the vector magnetic field intensity at the position of the detector 43. In this way the earth's field is automatically cancelled as well as any local field that has a gradient that would typically be much lower as compared to the gradient of the magnetic field intensity from the magnet 12. The end magnetic field detector 42 and the reference detector 43 should be able to detect a magnetic field intensity as low as 0.1 milligauss. The separation between the detectors 42 and 43 should be at least 2 cm and optimally between 5 and 10 cm. The magnetometer probe 41 could use any one of a variety of magnetic field detectors which could be Hall effect, fluxgate or giant magneto resistance (GMR) sensors. Of these, either the Hall effect or the GMR sensor is preferred because of their small size.

The electronics module and range indicator 46 would receive a signal from the magnetometer probe 41 that is indicative of the magnetic field intensity at the position of the end magnetic field detector 42. The output of the range indicator 46 could be an audio signal or a visual display or both. The audio signal could be an audio tone that was (for example) a higher pitched sound as the magnetic field detector 42 approached the magnet 12. Another audio detection means would be a succession of pulses that had a shorter time between pulses as the detector 42 gets closer to the magnet. When the detector is within a short distance from the magnet 12, the sound could become continuous. Once the tone was continuous, it is conceivable that, at that point, the pitch of the sound would become a higher frequency as the magnet 12 was approached. If a visual display was used to tell the surgeon that he/she was approaching the magnet 12, it could be (for example) an analog or digital meter that gives the approximate distance in millimeters or centimeters between the end of the probe 41 and the center of the magnet 12.

FIG. 4 also shows that the electronics module and range indicator 46 has a magnetic length adjustment knob 47 and a magnetic dipole moment adjustment knob 48. Each magnet 12 that is placed within a breast will have on its package a listing of the magnet's length and its magnetic dipole moment. In order to accurately determine the distance to the magnet 12, the range indicator 46 will have an adjustment for these two aspects of the magnet 12 that will correlate the range to that magnet 12 and the position of the probe 41. The knobs 47 and 48 will provide that adjustment. It is also envisioned that a standard magnetic marker could be used for all procedures thus obviating the need for having the adjustments made by the knobs 47 and 48.

A preferred embodiment of the magnet locating system 40 would use a wireless connection between the probe 41 and the range indicator 46, i.e., there would not be a wire 45 connecting the probe 41 to the range indicator 46. In this way, the surgeon could grasp the probe handle 44 and move it to seek the magnet 12 without being constrained in any way by the connecting wire 45. A wireless connection would allow the range indicator 46 to be placed at any convenient place in the operating room without being constrained by a connection to the wire 45. The magnet locating system 40 could be operated by either rechargeable or primary batteries. Not shown in FIG. 4 but required for the magnet locating system 40 are ON-OFF switches that turn the magnetometer probe 41 and the range indicator 46 either on or off. Also, a LOW BATTERY LIGHT is envisioned to tell the operating room personnel that new batteries are required. Still further, it is envisioned to have a volume control on the range indicator 46 to optimize the sound level desired by the surgeon during the procedure to excise the suspected tissue volume 30.

It is also envisioned to place all of the electronics of the system 40 within the handle 44 of the probe 41 so that there is no need for the wire 45 or the range indicator 46. Although a wireless connection could be accomplished between the handle 44 and the range indicator 46, having all of the electronics within the handle 44 provides the optimum system for detecting the location of the magnet 12. Thus it is envisioned that the handle 44 could contain all the electronic circuitry and an audio and/or visual means for correlating the position of the detector 42 with the position of any of the magnet markers described herein.

A removable sterile plastic cover (not shown) could be placed over the probe 41 including the handle 44 before the surgeon utilizes that magnetic field probe. This would better assure sterile conditions for the excision of the magnet 12 and the suspected tissue volume 30. Although the magnet injector 10 is a disposable device that is sterilized by the manufacturer, the magnet locating system 40 is not disposable and therefore a means must be provided for at least the probe 41 that is handled by the surgeon to be made sterile for every surgical procedure. The optimum means for attaining sterility is for a sterile cover to be placed over the probe 41 before the procedure. The range indicator 46 would not be covered but would not be touched by the surgeon during the procedure. The use of a wireless connection or having all the electronics within the handle 44 would make it easier to maintain sterility because there would be no connecting wire 45 that could compromise the required sterile conditions for this procedure.

When the magnet 12 is placed into the breast, it would be optimum to have it placed parallel to the patient's chest wall and for the surgeon to know the magnet's orientation so the detector 42 may be rapidly aligned with the implant. A standardized method of insertion either from right to left for the patient's right breast or from left to right for the patient's left breast can provide an optimum method for placement of the magnet 12 by routinely placing the long axis of the magnet 12 parallel to the chest wall and perpendicular to a line from the patient's head to the patient's feet. Alternatively, other orientations of the implant parallel to the chest wall may be equally successfully utilized if the implant orientation is demonstrated by the alignment of the points of a magnetic compass with the poles of the magnet 12 that may be observed while moving the compass over the marked breast, marked on the patient's skin after implantation, or pictorially described by the radiologist and communicated to the surgeon. With the prior knowledge of the orientation of the magnet 12, the surgeon using the magnet locating system 40 would immediately be able to orient the probe 41 to best locate the magnet 12. For example, if the preferred embodiment of a vector magnetic gradiometer is used, the detector 42 could be positioned to detect a magnetic field component from the magnet 12 implanted parallel to the patient's chest wall by simply aiming the probe 41 perpendicular to the body's long axis with the detector 42 aligned with the magnet 12. Such prior knowledge of the position of the magnet 12 would speed up the detection of its position within the breast.

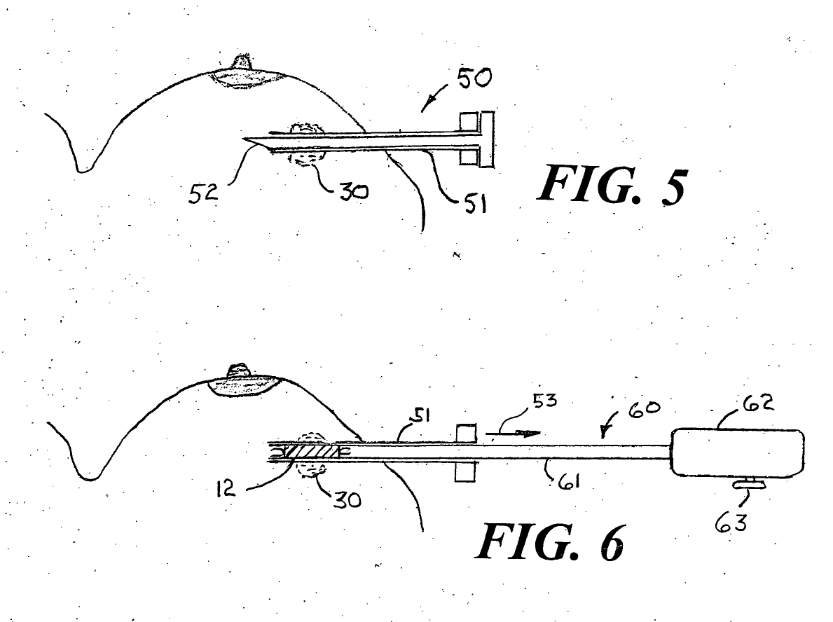

FIGS. 5-8 show an alternative method for placing the magnet 12 into the optimum position in a woman's breast. FIG. 5 shows a needle system 50 consisting of a needle cannula 51 with a square end into which a pointed stylet 52 has been placed. Although FIG. 5 shows a stylet 52 with an angled end point, a stylet 52 with a diamond shaped point can be at least equally effective in placing the needle system into the breast. Because the needle system is smaller and lighter and easier to handle as compared to the magnet injector 10 of FIG. 1, it could be advantageously used in certain circumstances to penetrate through the suspected tissue volume 30. As shown in FIG. 6, the next stage in placing a magnet 12 into the correct position within the suspected tissue volume 30 would be to use a magnet injector 60 to place the magnet 12 at the distal end of the cannula 51. This would be done after the stylet 52 was removed from the cannula 51. After the stylet 52 is removed from the cannula 51, the cannula 61 of the magnet injector 60 is then inserted so as to place the magnet 12 at the center of the suspected tissue volume 30. The cannula 51 is then slid back in the direction 53 to be completely out of the breast tissue. The magnet injector 60 is then used to pull back the cannula 61 while the magnet 12 remains correctly positioned within the suspected tissue volume 30. The injector 60 differs from the injector 10 in that its distal end is square and not pointed as is the shape of the distal end of the injector 10.

The needle system 50 may be made of MRI compatible materials (for example, titanium or non-magnetic stainless steel) thus allowing accurate positioning of the needle system 50 while the patient is within the strong magnetic field of an MRI machine and chamber. By first positioning a MRI compatible needle system 50 under MRI guidance, removing the patient from the strong magnetic field inside the MRI chamber and then using the magnet injector 60 outside of the MRI chamber, a magnet 12 may be accurately positioned without the need to ever bring the magnet 12 near the MRI magnetic field. In this fashion, the well-known hazard of projectile movement of a magnet 12 brought near the strong magnetic field used in MRI will be avoided.

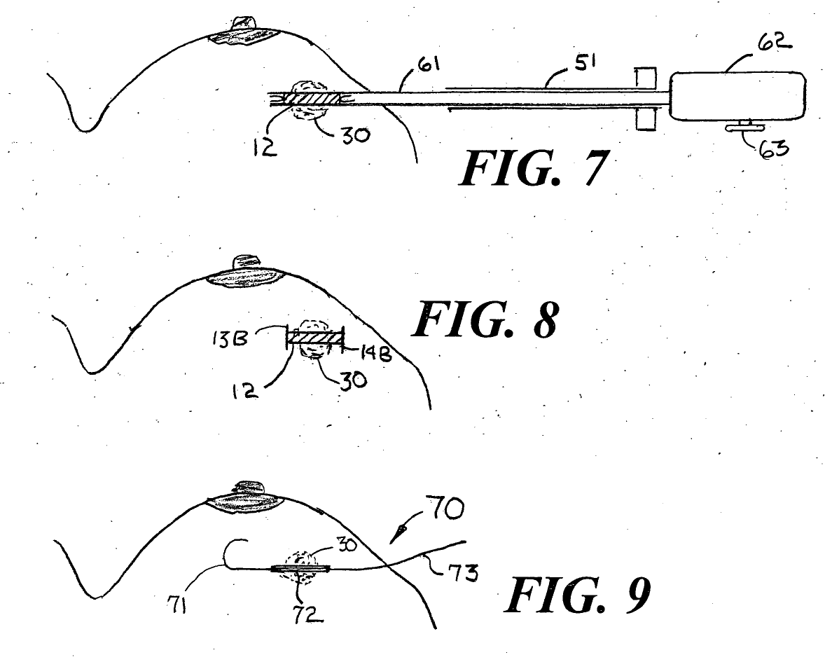

FIG. 7 shows the cannula 51 removed from the breast and slid onto the cannula 61 of the magnet injector 60. This is accomplished by holding the handle 62 of the injector 60 while pulling the cannula 51 out of the breast. The next step in this method for placing the magnet 12 through the suspected tissue volume 30 is to pull out the spring release handle 63 so that the spring (as shown in FIG. 1) pulls the cannula 61 out of the breast. The goal of having the magnet 12 with deployed anchors 13B and 14B placed accurately with the suspected tissue volume 30 is shown in FIG. 8. The magnet locating system 40 as shown in FIG. 4 can then be used by the surgeon to locate the magnet 12 within the woman's breast.

FIG. 9 illustrates an alternative embodiment of the present invention that uses a wire 70 with a hook 71 at its distal end and a proximal portion 73 that protrudes through the patient's skin. Except for the placement of a permanent magnet 72 at the center of the wire, this type of wire is what is typically used to mark a suspected tissue volume 30. The magnet 72 of FIG. 9 is shown without any tissue anchors that would be equivalent to the anchors 13B and 14B of FIG. 8. However, it should be understood that the magnet 72 could have either one or both anchors that are deployed like the anchors 13B and 14B of FIGS. 4 and 8. These anchors would prevent the axial migration of the wire 70 that does occasionally occur during localization. It should also be understood that the placement of such anchors would be a valuable improvement for the wire 70 even if the permanent magnet 72 was not used for localization. Although FIG. 9 shows that the wire 70 includes a hook 71, when anchors such as anchors 13B and 14B are used, the wire 70 could advantageously be straight and the hook 71 would not be needed to maintain accurate positioning of the magnet 72.

FIG. 10A shows an alternative embodiment of the magnet marker that is the pre-deployed magnet marker 80A within a cannula 84. This magnet marker 80A has main magnet bodies 81, connected by a narrow center flat 82 onto which two shape memory alloy center anchors 83A are attached in their pre-deployed state. The main magnet bodies 81 are a permanent magnet material such as Arnokrome 3 and the anchors 83A are preferably formed from the shape memory alloy Nitinol.

The pre-deployed magnet marker 80A of FIG. 10A is shown in FIG. 10B in its deployed state. FIG. 10B shows the deployed magnet marker 80B with the center anchors 83B in their deployed state. These anchors could be attached to the narrow center flat 82 by means of soldering, brazing or welding. For optimum biocompatibility the magnet 80B could be plated with a metal such as gold, platinum or any other metal that is biocompatible with human tissue. It is also envisioned to coat the magnet 80B with a biocompatible plastic such as Paralene.

FIG. 11A is another alternative embodiment magnet marker 85A shown in its pre-deployed state within a cannula 89. The magnet marker 85A has main magnet bodies 86, connected by a small diameter central cylindrical structure 87 and a sponge-like center anchor 88A. The center anchor 88A could be made from any biocompatible plastic material such as polyvinyl alcohol (PVA), or any bioabsorbable hydrophilic material such as denatured porcine gelatin (IE Gelfoam®), polyglycolic acid (IE Dexon®) or oxidized cellulose (IE Oxycel®; Surgicel®) or other similar materials well known to those versed in the art.

The pre-deployed marker 85A is shown in its deployed state in FIG. 11B. FIG. 1B shows the deployed magnet marker 85B with the center anchor 88B in its deployed state. Once this magnet marker 85B has been accurately placed, the center anchor 88B could be bioabsorbed without affecting the accurate positioning of the magnet marker 85B.

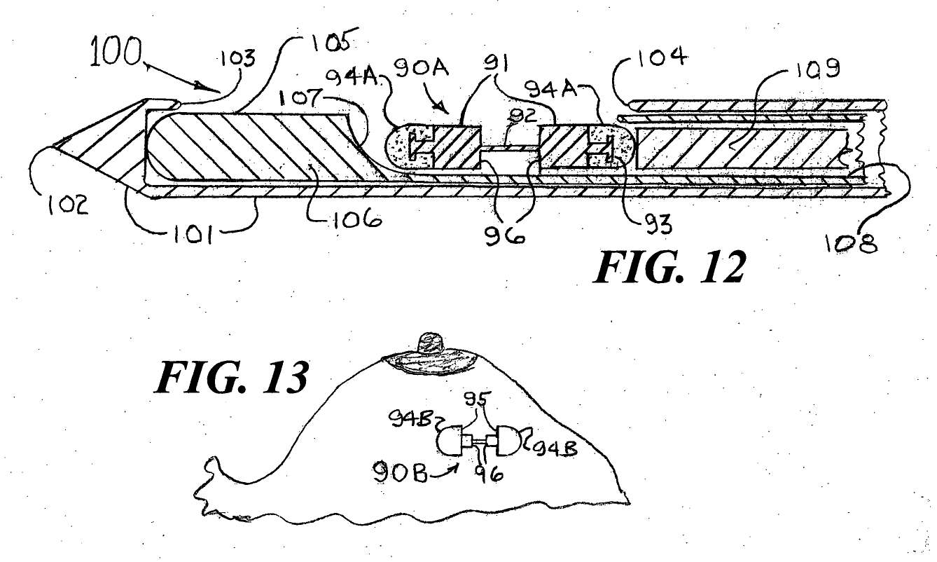

FIG. 12 is a longitudinal cross section of a distal portion of a biopsy device 100 that is shown after it completed a needle biopsy of suspected tissue in a female breast. After the biopsy is completed, a magnet insertion device 105 is placed within the biopsy device 100 for the purpose of placing the magnet marker 90A (shown in FIG. 12 in its pre-deployed state) to mark the region where the suspected tissue volume of breast tissue has been removed. The biopsy device 100 has a main body 101, a sharp distal point 102 a distal edge of its opening 103 and a proximal edge 104 of the opening in the biopsy device. The opening in the biopsy device 100 is first used to remove a suspected tissue volume of breast tissue from the female breast. Following the biopsy, the magnet marker 90A is delivered into the biopsy cavity. The tissue that has been removed is then examined by a pathologist to determine if there are any cancer cells. If the excised tissue sample shows no cancer cells, then the deployed marker 90B as shown in FIG. 13 remains within the breast tissue. If malignant cells are found in pathology, then the magnet marker 90B with an additional margin of breast tissue is excised from the patient's breast tissue. Since the deployed magnet marker 90B of FIG. 13 can be located by the surgeon using the magnetic probe system 40 of FIG. 4, an additional study to mark the location of the biopsy is no longer required.

As seen in FIG. 12, the pre-deployed magnet marker 90A has two main magnet bodies 91, a small diameter connecting wire 92 and two holding flanges 93 that retain expandable end sections 94A onto the magnet marker 90A. It should be understood that the magnet marker 90A is still another alternative embodiment of the invention of the magnet marker. The surfaces 96 of the marker 90A will help to anchor the magnet marker 90A after it is placed into a human breast as shown by the deployed marker 90B of FIG. 13.

The magnet insertion device 105 shown in FIG. 12 has a solid distal end section 106 with an inclined proximal surface 107 and a tubular portion 108 through which a push rod 109 is placed so that it can push the marker 90A into the region where the suspected tissue has been excised. After the biopsy device 100 has been used to collect the suspected tissue volume of the female breast, the rod 109 is used to push the magnet marker 90A in a distal direction against the inclined surface 107 that causes the magnet 90A to be pushed out of the biopsy device 100 and centered into the region where the suspected tissue was excised. The small diameter wire 92 must be flexible enough so that it will bend appropriately when the push rod 109 pushes the front section of the marker 90A against the inclined surface 107. Once placed into the breast (as shown in FIG. 13) the pre-deployed expandable end sections 94A become the expanded end sections 94B. As seen in FIG. 13, the expanded end sections 94B having surfaces 95 and the surfaces 96 of the main magnet bodies 91 all act as center anchors to hold the magnet marker 90B in place within the breast. The expandable end sections 94B can be formed from polyvinyl alcohol or any other expandable, sponge-like material that is known to be biocompatible.

Any of the magnet placement systems described herein can be used to place any of the different embodiments of the magnet markers into certain suspected tissue volumes within a human body. For example, the systems described herein could be used by a radiologist to place a magnet marker into a suspected tissue volume within a lung where a lesion is located that may be lung cancer. It is also envisioned to use the systems described herein at any location within a human body that is accessible by the injectors 10 or 60 for marking tissue that may be cancerous. Further, a magnet locating system 40 may be easily modified to allow for its placement on or through a thoracoscope, laparoscope or endoscope to allow the magnet locating system 40 to detect a magnet marker placed in any location within the human body and thus aid in the successful removal of such marked tissues during minimally invasive thoracoscopic, laparoscopic or endoscopic procedures. Further, the means and method of delivery of a magnet marker 90A via a flexible cannula as shown in FIG. 12 may be easily adapted by those of ordinary skill in the art for delivery following a biopsy performed during a thoracoscopic, laparoscopic or endoscopic procedure by inserting a long flexible cannula through the biopsy port of the instrument in order to mark a suspected tissue volume that may be cancerous at any location within a human body that is accessible by thoracoscopic, laparoscopic or endoscopic procedures.

Although the magnet 12 described herein is optimally in the form of an elongated cylinder, the cross section of the magnet 12 could have any shape including square, hexagonal, and octagonal, etc. For any such cross section, the largest transverse dimension can be considered to be the diameter of the elongated magnet 12.

Although a single magnet will be optimum for locating a typically small suspected tissue volume 30, it should be understood that two or more magnets could be used for localization of a large and/or complex suspected tissue volume 30. Although the optimum magnets for placement into the breast would have a length to diameter ratio of about 10 and a length of about 20 mm, it should be understood that a length to diameter ratio as small as 1.5 and a total length of only 3 mm could produce a sufficiently large magnetic field for detection by a magnetic probe. Furthermore, it should be understood that an optimum design for the magnet would be to have it plated with a metal that is both highly radiopaque and compatible with human tissue. An optimum plating would be gold, platinum or tantalum all of which are human tissue compatible and highly radiopaque.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A system for indicating the position of a suspected tissue volume within a human subject prior to a surgical excision procedure, the system including:
an elongated permanent magnet having a length of at least 3 mm and having a length between the magnet's north and south poles that is at least one and one-half times greater than the magnet's diameter, the elongated permanent magnet being adapted to be implanted through an insertion needle, the placement of the magnet being within the suspected tissue volume and the magnet further having an anchoring means, and
a magnet locating system having a probe to detect the magnetic field from the permanent magnet, the magnet locating system also having electronic circuitry that creates an audio signal and/or a visual display that indicates the distance from some point on the magnetic probe to the implanted magnet.

2. The system of claim 1 where the magnet is formed from a machinable metal alloy.

3. The system of claim 1 where the magnet has an anchor fixedly attached to at least one of its ends.

4. The system of claim 1 where the magnet has at least one anchor placed at or near the longitudinal center of the magnet or placed at one or both ends of the magnet.

5. The system of claim 4 where the anchor is formed from a shape memory alloy having a transition temperature that is just below body temperature.

6. The system of claim 4 where the anchor is formed from polyvinyl alcohol that expands after placement into the human subject.

7. The system of claim 1 where the shape of the magnet at or near its longitudinal center creates a center anchor for the magnet.

8. The system of claim 1 where the magnet is plated with a biocompatible metal that is also highly radiopaque.

9. The system of claim 8 where the plating on the magnet is gold.

10. The system of claim 1 where the suspected tissue volume is within the female breast.

11. The system of claim 1 where the suspected tissue volume is within a human lung.

12. The system of claim 1 where the probe of the magnet locating system is connected by a wire to the electronic circuitry that creates the audio signal and/or the visual display that indicates the distance of the probe from the implanted magnet.

13. The system of claim 1 where the probe of the magnet locating system has a wireless connection to the electronic circuitry that creates the audio signal and/or the visual display that indicates the distance of the probe from the implanted magnet.

14. The system of claim 1 where the probe contains all of the electronic systems including the range indication circuitry and audio and/or visual range indication means.

15. The system of claim 1 including a sterile cover that can be placed over the probe to assure the sterility of the probe during the surgery to remove the suspected tissue volume.

16. The system of claim 1 where the audio signal indicates the distance from the probe to the implanted magnet by the changing pitch of the audio signal.

17. The system of claim 1 where the audio signal indicates the distance from the probe to the implanted magnet by the time between sound pulses.

18. The system of claim 1 where the audio signal indicates the distance from the probe to the implanted magnet by the time between sound pulses and also a changing pitch of the sound as the probe closely approaches the magnet.

19. The system of claim 1 where the visual indication is a meter that indicates the distance from the probe to the implanted magnet.

20. The system of claim 1 further including a magnet injector that is designed to accurately place the magnet within the suspected tissue volume.

21. The system of claim 20 where the magnet injector has a handle into which a cannula is situated, the cannula having a distal end from which the elongated permanent magnet is advanced into the region where the suspected tissue of the human subject was removed.

22. The system of claim 21 where inside the handle of the magnet injector there is a rod that is used to push the magnet into the region where the suspected tissue was removed.

23. The system of claim 1 where the magnet delivery system is adapted for placement using an instrument that is selected from the group consisting of a thoracoscope, laparoscope and endoscope.

24. The system of claim 1 where the probe of the magnet locating system is adapted to be fixedly attached at or near the end of an instrument that is selected from the group consisting of a thoracoscope, laparoscope and endoscope.

25. A system for indicating the position of a suspected tissue volume within a human subject following a large needle biopsy procedure, the system including:
an elongated permanent magnet having a length of at least 3 mm and having a length between the magnet's north and south poles that is at least one and one-half times greater than the magnet's diameter, the elongated permanent magnet being adapted to be implanted through a biopsy needle and the placement of the magnet being within the suspected tissue volume within the human subject; and
a magnet locating system having a probe to detect the magnetic field from the permanent magnet, the magnet locating system also having electronic circuitry that creates an audio signal and/or a visual display that indicates the distance from some point on the magnetic probe to the implanted magnet.

26. A method for indicating the position of a region within a human subject from which a volume of tissue that was suspected of being malignant was removed by means of a needle biopsy, the method including the following steps:
a) performing a needle biopsy procedure to remove tissue from a human subject which tissue is suspected of being malignant; and
b) using a magnet injector to implant an elongated permanent magnet into the region where the suspected tissue volume was removed.

27. The method of claim 25 further including an additional step of detecting the magnetic field of the permanent magnet by means of a magnet locating system that has a probe that can detect a magnetic field and also has electronic circuitry that creates an audio signal and/or a visual display that indicates the distance from some point on the magnetic field probe to the implanted magnet and then performing still another step of surgically removing the magnet and the tissue volume surrounding the magnet from the human subject.
